(19) United States
(12) Patent Application Publication
(10) Pub. No.: US 2004/0267234 A1
(43) Pub. Date: Dec. 30, 2004

(54) Implantable ultrasound systems and methods for enhancing localized delivery of therapeutic substances

(76) Inventors: Gill Heart, Tucson, AZ (US); Gideon Tolkowsky, Herzliya (IL); Axel Brisken, Freemont, CA (US); Joe Karratt, Millbrae, CA (US)

Correspondence Address: KIRKPATRICK & LOCKHART LLP, 75 STATE STREET, BOSTON, MA, 02109-1808, US

(21) Appl. No.: 10/746,311

(22) Filed: Dec. 24, 2003

Related U.S. Application Data
(60) Provisional application No. 60/463,623, filed on Apr. 16, 2003.
(60) Provisional application No. 60/470,585, filed on May 15, 2003.

(51) Int. Cl. A61M 31/00

(52) U.S. Cl. 604/500; 604/22

(57) ABSTRACT

Implantable ultrasonic transducer devices and methods are provided to enhance local delivery and tissue uptake of therapeutic substances using phonophoresis. The transducers are adopted to be implanted immediately adjacent or within the target tissue to which the therapeutic substances also are delivered.

This application claims benefit of Provisional Applications No. 60/463,623, filed Apr. 16, 2003, and 60/470,585, filed May 15, 2003.

### FIELD OF THE INVENTION

This invention relates to implantable ultrasound transducer devices and their use in enhancing delivery of therapeutic substances to tissue by phonophoresis.

### BACKGROUND OF THE INVENTION

Most medicinal, pharmacological and other therapeutic substances are delivered systemically by swallowing, breathing, injection or intravascular delivery. The substance ultimately reaches the vascular system and is transported to tissue and organs throughout the body. However, in cases where the targeted clinical disorder is localized, systemic methods may present some disadvantages. In order to create a sufficiently high concentration of the substance at the target site, systemic administration requires high dosage in comparison to the amount actually required at the target site. Exposure of untargeted organs or tissues may cause undesirable side effects. Moreover, in some disorders, such as those involving the neurological system, systemic delivery can fail due to the inability to deliver an adequate quantity of the substance across a biological barrier such as the blood-brain barrier.

It is important to deliver the substance to the desired target in a therapeutically effective dose over a period of time, while limiting or avoiding systemic or collateral risk to the patient. For orally administered substances, delivery obstacles include overcoming the degenerative effect of the gastrointestinal system, high level filtration by the liver, side effects due to the systemic effects of the substance and low amounts of administered substances reaching intended target. Each of subcutaneous, intravascular, intravenous and mucosal delivery presents risks of overdose, infection and the side effects of a systemic approach. The risks and difficulties inherent in systemic substance delivery have been long recognized, as evidenced by numerous examples of local substance delivery systems, such as the use of topically applied substances, local delivery of therapeutic substances internally of the body, as by implantable infusion pumps to deliver therapeutic substances to a specific organ, and encapsulated therapeutic materials adapted to degrade and release the substance at the specific target site.

However, even when a substance is released locally at the treatment site, the rate at which it is taken up by the targeted tissues and cells may limit the effectiveness of the treatment. The rate of substance uptake can play a critical role. One such example is in treatment of glioblastoma, a particularly aggressive form of brain cancer in which the tumor may typically double in mass in approximately eleven days. Treatment for glioblastoma involves immediate surgery to remove the tumor from the brain. However, because removal of excess tissue about the peripheral margins of the tumor may damage healthy brain cells, the surgeon may be reluctant to excise such peripheral tissue. Instead, upon removal of the tumor, the resulting cavity may be filled with a chemotherapeutic substance intended to diffuse into the peripheral tissue including cells and extracellular matrix, to treat cancer cells that may have diffused beyond the resected volume. One such chemotherapeutic substance is available commercially from Guilford Pharmaceuticals under the trade designation Gliadel wafers. Gliadel wafers are provided in the form of small, dime-sized biodegradable biopolymer that delivers a chemotherapeutic drug (polifeprosan 20 with carmustine) directly to residual tumor cells after the tumor has been resected. Up to eight Gliadel wafers may be implanted along the walls and floor of the cavity left after the tumor has been resected. The wafers dissolve slowly, releasing the drug and bathing the surrounding cells. Transport of the chemotherapeutic agent relies on the body's natural diffusion mechanism, a passive process.

Although reliance on a passive, natural diffusion process of a locally placed substance, may be more effective than systemic treatment, it nevertheless presents a number of difficulties in that (i) only small size molecules will get into cells and their internal organs, (ii) substance uptake rate by cells is limited to the natural diffusion rate and (iii) the drug may have difficulty in crossing extracellular matrix to reach distant cells.

Other recent clinical treatment methods have involved the use of therapeutic ultrasound. Therapeutic ultrasound operates in the range of about 20 KHz to about 10 MHz. This frequency range may be subdivided according to the resulting physical biological effects. At the upper end of the frequency range, a focused high intensity ultrasound beam causes heating and leads to tissue and cell necrosis. Such high intensity focused ultrasound is the most common use of therapeutic ultrasound. Therapeutic ultrasound, at the lower frequencies (e.g., about 20 KHz to about 2 MHz) has been used to enhance substance delivery by using the effects of phonophoresis to enhance the uptake of the therapeutic substance through and into tissue. Such phonophoretic use of ultrasound typically applies the ultrasound from a source outside of the body, directing the ultrasound waves transdermally, with the therapeutic substance being placed topically, as by application of a skin patch, to pass through the skin and into internal tissue. Using some portions of the lower frequency range of therapeutic ultrasound takes advantage of the cavitation phenomena as explained in “An Experimental and Theoretical Analysis of Ultrasound-Induced Permeabilization of Cell Membranes,” J. Sundaram, B R. Mellein, S. Mitragotri, Biophysical Journal Vol. 84, pp. 3087-3101, 2003 and Miller et al in “A Review of in Vitro Bioeffects of Inertial Ultrasonic From a Mechanistic Perspective”, Ultrasound Med.Biol. 1996 22:1131-1154. Organ permeability has been explained as being mediated by oscillation and collapse of gaseous cavities, a process referred to as cavitation, as described by Leighton in “The Acoustic Bubble”. Academic Press, San Diego 1997 or by Lokhandwalla and Sturtevant in “Mechanical Haemolysis in Shock Wave Lithotripsy”. Phys. Med. Biol. 2001 46:413-437.

While the precise mechanism by which phonophoresis improves cellular uptake is not well understood, it is theorized that it affects several levels of cell structure. Among the theories offered to explain the increased uptake in that the oscillatory collapse of the gaseous bubbles temporarily increases the permeability of neighboring cells and extracellular matrix and affects intracellular components to make them more permeable to the molecules of the therapeutic substance. As a result, molecules of therapeutic substance may penetrate into cells and their sub-cellular components at a faster, more efficient rate. Phonophoresis has been used clinically for transdermal drug delivery applications as described in ‘Synergistic Effect of Enhancers for Transdermal Drug Delivery’, S Mitragotri, Pharmaceutical Research, Vol. 17, No. 11, 2000.

In another approach, proposed in ‘Where a Pill Won't Reach’, by Robert Langer, Scientific American, April 2003, pp 33-39, a minute volume of a drug is enclosed in polymer-based, ultrasound-sensitive microspheres. The microspheres are injected into and travel with the blood stream. When the micropheres reach the region of the target organ, the encapsulating polymer is ruptured by an ultrasound wave generated and directed from outside the body toward the target organ. The drug thus is released and, hopefully, diffuses from the blood vessel into the target organ at the natural diffusion rates for the particular tissue. This alternative relies upon passive diffusion of the substance into the targeted organ. Additional theories attempting to explain the phenomenon of phonophoresis have been based on the suggestion that acoustic radiation pressure might facilitate movement of molecules through the tissue medium. Other theories suggest acoustic streaming as a mechanism for circulating fluid substances around structures of differing acoustic impedance. Still other theories suggest that the mere presence of the acoustic field results in membrane permeabilization due to the rapid and relatively large amplitude stretching and compression of cellular structures. As used herein, the term “phonophoresis” is not intended to be limited to any particular theory by which the phenomenon may be explained.

If the target organ is deeply embedded in the body, the ability to develop a phonophoretic effect at the target site to facilitate substance uptake is severely limited. Due to physical limitations the effectiveness of the phonophoresis process declines as a function of tissue depth. In the case of the brain, yet another physical limitation to applying ultrasound is the high attenuation of the ultrasound beam by the human skull.

Also among the difficulties encountered with existing local substance delivery devices is that they involve delivery only of relatively small molecule drugs. Certain molecules often referred to as ‘smart molecules’ (RNAi, sRNA, double strand RNA) presently are considered as candidates for treatment of various diseases. These smart molecules, although considered as medium-sized, still are too large to diffuse passively through the extracellular matrix and penetrate into cells and intracellular components. Although viruses may be usable to transport such smart molecules into cells, the use of viruses as carriers presents potential risks. Another alternative to viral delivery of genes is via microspheres, which are tiny gas-filled or drug-filled ultrasound-sensitive, synthetic polymer spheres used to deliver drugs to specific sites, such as tumors. It has been proposed to use ultrasound to rupture such microspheres, causing them to release their contents and diffuse passively, as described in U.S. Pat. No. 5,580,575. This type of treatment, however, is not believed to be clinically available at present.

In the emerging field of gene-therapy, the genetic agents typically are of high molecular weight and must be delivered locally rather than systemically. Gene therapy delivery methods involve, among other things, the use of microspheres and liposomes activated to release a substance by externally applied ultrasound. Difficulties in localized delivery and adequate passive uptake of the genetic agents constitutes a severe limitation in the clinical exploitation of gene therapies.

Thus, despite recent advances, there is a continuing need for improved sustained, local substance delivery methods and technologies which can provide a more targeted delivery of therapeutic substances as well as improved and regulated uptake of the substance by the targeted tissue and while reducing the incidence or severity of side effects. There also exists a need for improving the delivery and uptake of medium- and large-sized molecule substances.

### SUMMARY

In one of its aspects, the present invention involves implantation of an ultrasonic transducer adapted to generate ultrasound energy in the lower range of ultrasound frequencies, in a patient's body in close proximity both to the substance to be delivered and to the targeted tissue or organ intended to be treated by the substance. The transducer is arranged and oriented to direct lower frequency ultrasound to the target. The therapeutic substance may be contained in a reservoir that may be incorporated into or may be separate from the implanted device. When separate, the reservoir may be implanted or may be located externally of the patient's body.

In another aspect of the invention therapeutic substance is delivered from an outlet that is within or immediately adjacent the target tissue or organ. This is of particular importance in connection with therapeutic substances that have a relatively short half-life in which it is important to advance the substance into the target tissue and cells as quickly as possible.

The ultrasound device may be implanted simultaneously with the therapeutic substance or may be implanted strategically in a position such that the therapeutic substance may be delivered, separately, to the target region, at which time the ultrasound energy can be applied to the target tissue to enhance uptake of the substance. For example, if the target region is in proximity to an accessible blood vessel sufficiently close to the target, the ultrasound transducer may be implanted in close proximity to the target region. Ultrasound-sensitive substance-encapsulating microspheres then may be injected into the blood stream upstream of the target region at a location at which the microspheres will flow through or adjacent the target region. As the microspheres flow to the target region, the implanted ultrasound transducer can be operated to activate the local release of the encapsulated substance as it passes through the ultrasound field and simultaneously enhance the uptake of the released substance by the vessel wall and the surrounding or adjacent targeted tissue.

In yet another therapeutic configuration, the ultrasound device may be implanted together with the therapeutic substance. For example, in the case of a resected brain tumor, the transducer may be implanted in the brain immediately adjacent to implanted bio-absorbable, therapeutic wafers. The implanted ultrasound system is activated to induce phonophoresis in the brain tissue enabling faster and more efficient localized uptake of the drug released by the wafers. The wafers may be considered to provide a reservoir of therapeutic substance to be applied to the tissue upon release.

In other applications of the principles our invention, the implantable ultrasonic transducer is used in combination with an implantable substance reservoir adapted to hold a flowable form of therapeutic substance. The implantable ultrasonic transducer is positioned within or in immediate proximity to the targeted organ, tissue or cells. The reservoir, which contains the therapeutic substance, may be encapsulated and self-contained together with the transducer as an integral device, or may be separately provided and positioned in the body within or in close proximity to the targeted organ, tissue or cells and the transducer. The flowable therapeutic substance may be emitted through outlet ports that may be disposed in immediate communication with the reservoir. The reservoir also may be implanted separately in a remote location and may be connected by an umbilical cord having one or more outlets to deliver the therapeutic substance at the region of the target tissue. The therapeutic substance should be delivered from an outlet at or in the immediate vicinity of the target site.

In another aspect of the invention a reservoir is provided in association with a pump mechanism for delivering flowable therapeutic substance to an outlet located to deliver the substance directly to the target tissue. In various aspects of the invention the pump may be incorporated into the ultrasound device in an integrated unitary implant or may be implanted at a remote location in the body and connected to the outlet through an umbilical cord. In other aspects of the invention the pump may be located externally of the patient and connected with an umbilical cord to the outlet.

The implantable ultrasound device may be shaped as clinically desired. For example, in some applications an ultrasound device may be adapted to generate ultrasound waves in a generally spherical, omnidirectional, unfocused pattern. Transducers having shapes other than spherical also may be employed.

In another aspect of the invention, the device and methods use ultrasound energy at levels that will not adversely affect the viability of the tissue and cells which are exposed to ultrasound. In particular, the levels of ultrasound energy employed in the practice of the invention are such that no substantial tissue necrosis occurs. Tissue necrosis would interfere with the objectives of the invention, to enhance the tissue uptake of therapeutic substances so that those substances can have the desired biological effect on living tissue.

The device may be operated by electronic control components that may be self contained in a single, integrated implantable device or may be implanted separately and connected by an umbilical cord. The electronic control elements also may be located externally of the patient and connected to the implanted components by an umbilical member. When the therapeutic substance is in a flowable form, the system also may include a pressure source for urging the flowable therapeutic substance from the reservoir to and through the outlet into contact with the tissue. In various embodiments, the pressure source may be integral with the implanted transducer, may be located externally of the body, or may be implanted at a location remote from the transducer and umbilically connected to the reservoir.

The electronic control components enable variation in the substance delivery and ultrasound protocols. They enable variation in the delivery rate as well as any other variable parameter associated with the pumping system for delivering a flowable therapeutic substance. The ultrasound may be controlled to switch the transducer on and off, to vary the intensity of the ultrasound field developed by the transducer by varying the amplitude of the exciting signal, to vary the duty cycle in which the ultrasound transducer is operated, and to vary the number of times per day that the ultrasound device is operated.

The foregoing controls may be operated through a computer and enable wide variation in control of the parameters. In particular, it permits a patient's treatment regimen to be changed if, upon monitoring the patient, a change in the treatment is indicated.

The invention also contemplates various methods, including surgical implantation of an ultrasound transducer in immediate proximity to the target tissue and oriented to direct lower range frequency ultrasound toward the target tissue to induce phonophoresis from a location internally of the patient. Another aspect of the method of the invention involves application of therapeutic substance directly to the immediate region of the target tissue to enable phonophoresis to enhance tissue uptake of the substance substantially immediately. In still another aspect of the invention small, large or medium size molecules may be phonophoretically diffused through tissue and cellular membranes by application of ultrasound energy at a frequency and energy level at which no substantial tissue necrosis occurs, and at which the ultrasound energy is applied from a location immediately adjacent the target tissue.

### THE DRAWINGS

The various aspects invention, their objects and advantages, will be appreciated more fully from the following description of several illustrative examples considered together the accompanying drawings wherein:

FIG. 1 is a somewhat diagrammatic sectional view of an implantable ultrasound transducer as may be used in the practice of the invention.

FIG. 2 is a diagrammatic illustration of an embodiment of the invention as it may be implanted within a surgically formed cavity in the brain of a patient for treatment of surrounding tissue about the remaining region of a resected tumor.

FIG. 3 is a schematic diagram of the elements of an embodiment of an integrated implantable substance delivery device;

FIG. 4 is a diagrammatic sectional illustration of a spherical embodiment of a device embodying principles of the invention;

FIG. 5 is an enlarged illustration of a one-way valve port through which the therapeutic substance can be delivered from an implanted reservoir to the target region;

FIG. 6 is a sectional illustration of the valved port of FIG. 5;

FIG. 7 is a diagrammatic sectional illustration of the connector end of an umbilical cord by which electrical signals and fluid can be delivered to the implantable device;

FIG. 8 is a diagrammatic illustration of a pair of hemispherical transducers adapted to be assembled in a spherical shell;

FIG. 9 is a diagrammatic illustration of an assembled spherical device having an internal ultrasound-absorbing member.

FIG. 10 is a diagrammatic sectional illustration of an embodiment of an ultrasound substance delivery device embodying principles of the invention; and

FIG. 11 is a diagrammatic sectional illustration of another embodiment of an implantable ultrasound device having an air backing and in which the source of therapeutic substance is separate from the transducer;

FIG. 11A is a diagrammatic illustration of a separated implantable reservoir for a flowable therapeutic substance;

FIG. 11B is a diagrammatic sectional illustration of another embodiment of a combined transducer and reservoir in which the reservoir is disposed about the transducer;

FIG. 11C is a diagrammatic sectional illustration of a portion of the transducer of FIG. 11B illustrating its layers;

FIG. 11D is a diagrammatic sectional illustration of a portion of the reservoir of FIG. 11B illustrating outlets defined by valved ports;

FIG. 11E is a diagrammatic sectional illustration of an alternate configuration for the reservoir illustrated in FIG. 11B in which the reservoir is formed as a thin membrane having micropores;

FIG. 12 is a diagrammatic sectional illustration of another embodiment of a device incorporating the principles of the invention;

FIG. 12A is an enlarged diagrammatic illustration of the module shown in FIG. 12;

FIG. 13 is an illustration of a piezo composite device in a partial state of fabrication in which a number of piezoelectric elements are contained in a polymer in a spaced array;

FIG. 14 is a sectional illustration of part of the piezo composite device of FIG. 13 with common ground and signal electrodes connecting the piezoelectric elements;

FIGS. 15A-15E depict the fabrication of a spherical piezo composite device; and

FIGS. 16A-16E depict another technique for fabricating a spherical piezo composite transducer; and

FIG. 17 is a block diagram illustrating the relation of the electronic controls.

### ILLUSTRATIVE EMBODIMENTS

FIG. 1 illustrates an implantable ultrasound transducer assembly 10 as may be used in practicing the phonophoretic aspects of the invention. The transducer assembly 10 includes a piezoelectric layer 12, a pair of conductive electrode layers 14, 16 overlying opposite faces of the piezoelectric layer 12 and defining the poles of the transducer, a pair of conductors 18, 20 connected to electrodes 14, 16, an acoustic matching layer 22 and a layer of biocompatible material 23 to encapsulate the components. The conductors 18, 20 are housed in an umbilical cord 25 formed from materials that provide electrical insulation and assure biocompatibility as will be familiar to those skilled in the art of implantable devices. The end of the cord 25 may be connected directly to a controllable power source or may have a connector 27 by which the conductors 18, 20 can be coupled to a source of electrical signals to activate the transducer. The device shown in FIG. 1 will emit ultrasound energy in opposite directions along the directions of transducer thickness as suggested by the arrows 11. While the implantable ultrasound transducer assembly 10 is depicted in a flat configuration in FIG. 1, it may be formed in any variety of shapes, such as spherical, among others, depending upon the needs of the particular application. The beam profile of the transducer may be varied by varying the shape of the radiative surface. The connector 27 may be implanted just beneath the skin or may be disposed externally. The connector may be coupled to a controllable source of signals, as by hard wired connectors. Other means for operatively associating the connector 27 with a source of operating signals may be provided. Additionally, an inductive circuit may be substituted for the connector by which signals may be induced, from a location external of the patient, to excite the transducer. The device as shown in FIG. 1 is an ultrasound-only device that may be placed independently of the mode of delivery of the therapeutic substance to the target tissue.

The thickness of the ceramic piezoelectric layer 12 typically may be the order of one half of a wave length and the matching layer may be of the order of one quarter of a wavelength and selected from a material having the correct acoustic impedance, as is familiar to those skilled in the art. The layer of biocompatible material 23 should have an acoustic impedance close to that of human tissue and may include material such as silicone rubber, polyethylene and polypropylene, among others. While it may be possible to use biocompatible materials having a higher acoustic impedance, that should be compensated for by appropriately varying the thickness of the biocompatible layer, as by making the layer thinner.

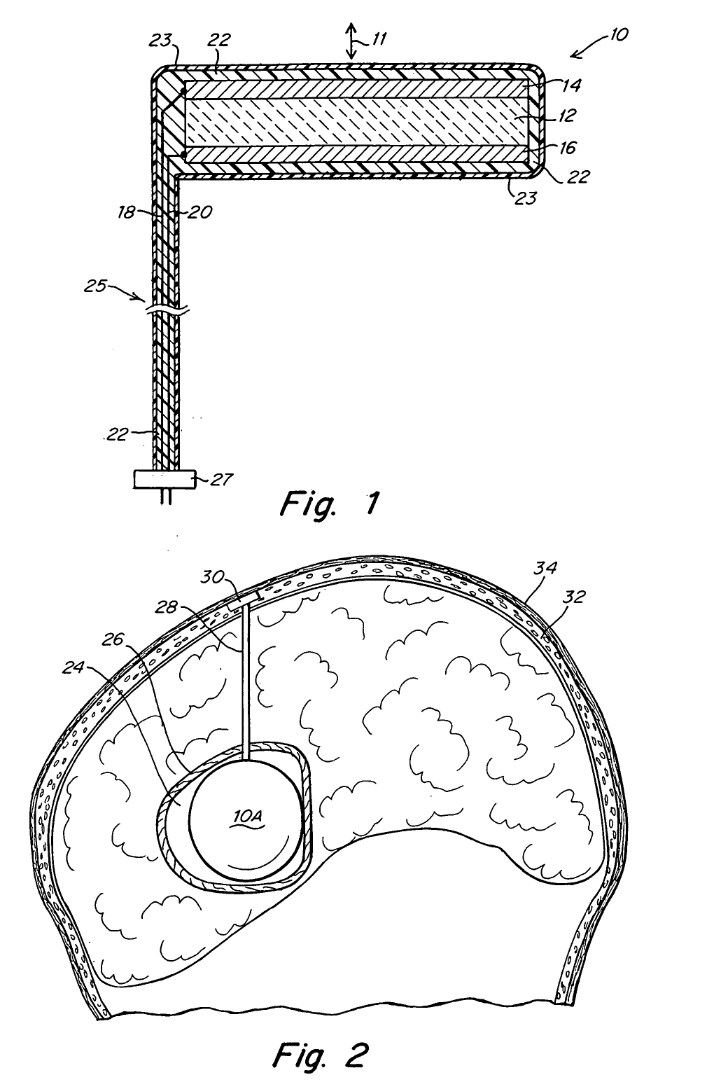

FIG. 2 illustrates, diagrammatically, one way by which the invention may be practiced, in the context of treatment for a brain tumor. By surgical removal of as much of the tumor as is considered appropriate, a cavity 24 will have been formed in the brain tissue. Because of the desirability of minimizing the loss of functioning brain cells, the neurosurgeon may be expected to leave some residual tumor 26. In one mode of practicing the invention, an implantable ultrasound transducer device 10A, shown in this illustration as spherical, is implanted in the cavity 24 to be in close proximity to the residual tumor 26 and surrounding tissue. The device should have a shape and ultrasound characteristics selected as suitable for the particular anatomy of the tumor or other type of treatable tissue and the resulting surgical configuration. The configuration and characteristics of the implanted device and transducer are selected so that it can be operated to generate and direct ultrasound toward the target tissue with intensity sufficient to penetrate the tissue to cause phonophorisis to a desired depth in the tissue. In the embodiment illustrated diagrammatically in FIG. 2, in which the device 10A is implanted within a cavity remaining after resection of a brain tumor, the device may be spherical so as to generate and direct ultrasound waves in an omnidirectional pattern and with an intensity sufficient to generate a phonophoretic effect to a desired radius. The radius should be sufficient to include all residual tumor 26 as well as some surrounding tissue as determined by the physician, in order to include cells that may have begun to migrate. The thickness of tissue to be treated may range from microns to centimeters.

The implantable device 10 may include an umbilical cord 28 by which electrical signals can be transmitted from a source to the internal piezoelectric transducer to generate and control operation of the device. The umbilical cord 28 may terminate in a portal 30 to provide electrical access to the cord 28. The portal may be placed subcutaneously on the patient's skull 32, as shown, or may be positioned externally of the body, with the cord 28 protruding through the skull and the scalp 34. It may be noted that a cavity surgically formed in brain tissue will tend to close about the implanted device. Additionally, voids that may initially exist in the cavity 24 between the brain tissue and the device will be filled by cerebro spinal fluid providing a void-free medium for ultrasound transmission.

The therapeutic substance that is to be applied may be placed within the cavity 24 by various means. In one approach, the therapeutic substance may be placed surgically and directly in the cavity 24 together with the ultrasound assembly 10. In other modes of operation, a reservoir containing the substance may be placed in the cavity and may be configured to dispense the therapeutic substance in a controlled manner. The device may comprise at least one implantable reservoir capable of controllably dispensing a desired quantity of a substance to the extracellular matrix or the targeted organ, tissue, or cell and an implantable transducer capable of generating ultrasound energy sufficient to produce the desired level of phonophoretic effect in the tissue at the target region. The reservoir may be configured to be replenishable with therapeutic substance, for example, by incorporating into the umbilical cord 28 a lumen adapted to deliver the therapeutic substance from the portal 30 to the reservoir. The reservoir also may be incorporated into the implantable assembly as an integral component, as will be described. In other embodiments, as when the target region is very small, the reservoir may be implanted in a location remote from the dispensing outlet and transducer, with an umbilical cord connecting the reservoir and dispensing outlet. As will be described the control electronics and power source may be implanted independently of the other components of the system.

The term “substance” as used herein is meant to include all manner of compositions for which local delivery could be employed. Such compositions may include, but are not limited to, chemotherapeutic compounds, genetic material, drugs, vitamins, amino acids, peptides and proteins, nucleic acids, DNA or RNA, anti-fungal agents, antibiotics, hormones, vitamins, anti-coagulation agents, antivirals, anti-inflammatories, local anesthetics, radioactive agents, organic and inorganic compounds, contrast agents, therapeutic agents with short-life cycle, bubble nuclei, micro-spheres (substance encapsulated), combinations thereof and the like. The substance may be in a fluid or fluent form, selected to have a viscosity appropriate to the flow and delivery requirements of the particular application, or may be in the form of surgically implantable biodegradable biopolymer or the like containing the therapeutic substance.

As used herein, the term “reservoir” is intended to include any device for containing or carrying a substance. For example, the reservoir may include a walled container adapted to hold a fluid or fluent substance such as saline, alcoholic saline or protein-buffered saline carrying the therapeutic substance. The reservoir may contain a substance contained in a hydrogel, where the hydrogel may be made of materials that are well known in the art such as synthetic polymers, including but not limited to, simethicone, silica gel, silica rubber, polyvinyl alcohol, polyethylene glycol, polymethacrylate, polypropyleneglycol, copolymers and derivatives with and without cross-linking and other polymers such as alginic acid, pectins, albumin, collagen, and other materials suitable for forming a gel to contain the desired substance. Similarly, the reservoir may be in the form of a synthetic, biodegradable, solid polymer such as PCl, (20:80) PLCl, PGLCl, PLA, or combinations thereof containing the therapeutic substance.

In most applications of the invention, it will be desirable to deliver the therapeutic substance directly to the immediate region of the target site, either by implanting the therapeutic substance at the site or delivering it through a delivery system directly to the site. This is particularly important with those substances that may have a relatively short half-life, possibly of the order of several minutes, and must be taken up by the tissue very quickly.

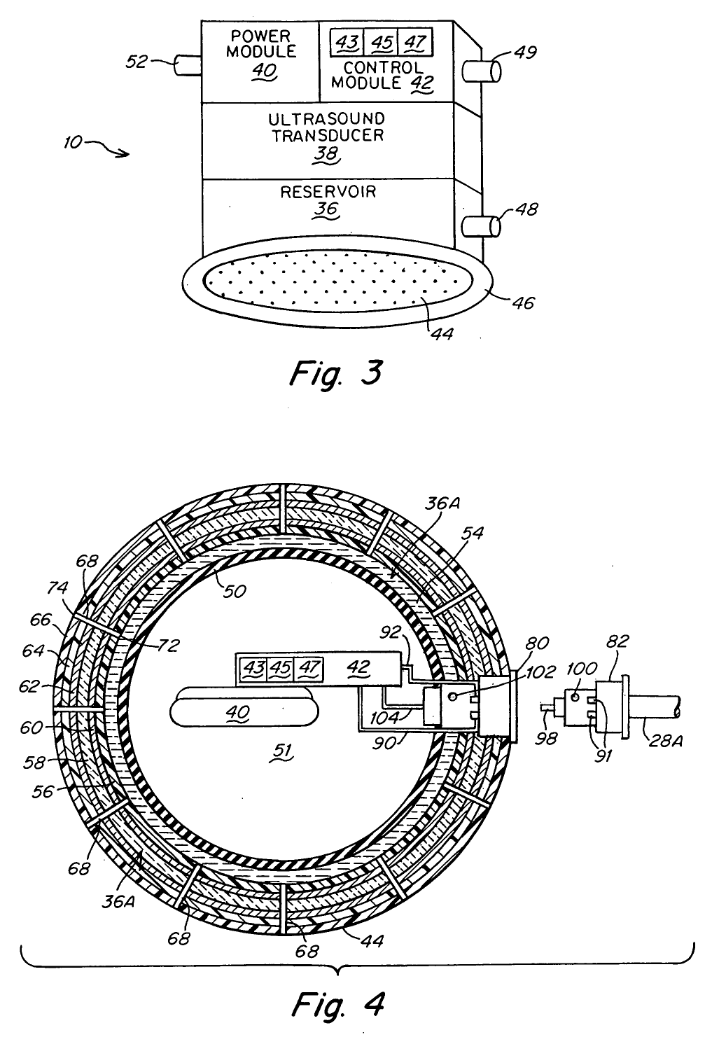

FIG. 3 illustrates generally, in block diagram form, the components of an integrated implantable device that may be used in the practice of the invention. The device may be considered as having several modules including a reservoir 36 for therapeutic substance, an ultrasound transducer 38 assembly, a power module 40 and a control module 42. The device also includes an outlet surface 44 having outlets that are fluidly coupled with the reservoir 36 and through which therapeutic substance contained in the reservoir 36 may be delivered to the target tissue. Depending on the configuration of the device, it also may include an attachment module 46 by which the device may be secured in place, as by suturing or the like to tissue or bone.

The reservoir 36 may include a refill and evacuation port 48 through which therapeutic substance can be removed from or delivered into the reservoir. The size and configuration of the refill port 48 should be based on substance type and anatomical location of the implanted device. In the case of a deeply implanted device, such as that shown in FIG. 4, the umbilical cord 28A may be coupled at one end 80 to the port 48 and extend to a location near the skin, such as the scalp 14, with the access portal 30 attached to the other end of the umbilical cord. Portal 30 may be positioned beneath or extend through the skin 14 to provide for direct coupling of a substance refill, a pressurization source, or electrical connections for power, data and signal transfer.

The control module 42 may include an electrical communication port 50 and the energy module 40 may include a port 52, for example, to enable recharging of a battery energy module. Substance release from the reservoir 36 may be controlled and regulated, in some embodiments, by selectively pressurizing the substance contained in the reservoir 36. By selectively modifying the flow through the outlets 44, the dose or rate of substance dispensed from the reservoir 36 may be controlled.

Selecting and controlling the viscosity of a suitable substance carrier such as the above-described hydrogel also may affect throughput of the substance through the outlet surface. The precise determination of these characteristics or parameters is predicated on variables such as the selected substance, the characteristics of the target, and the clinically effective dosage of the substance, among others.

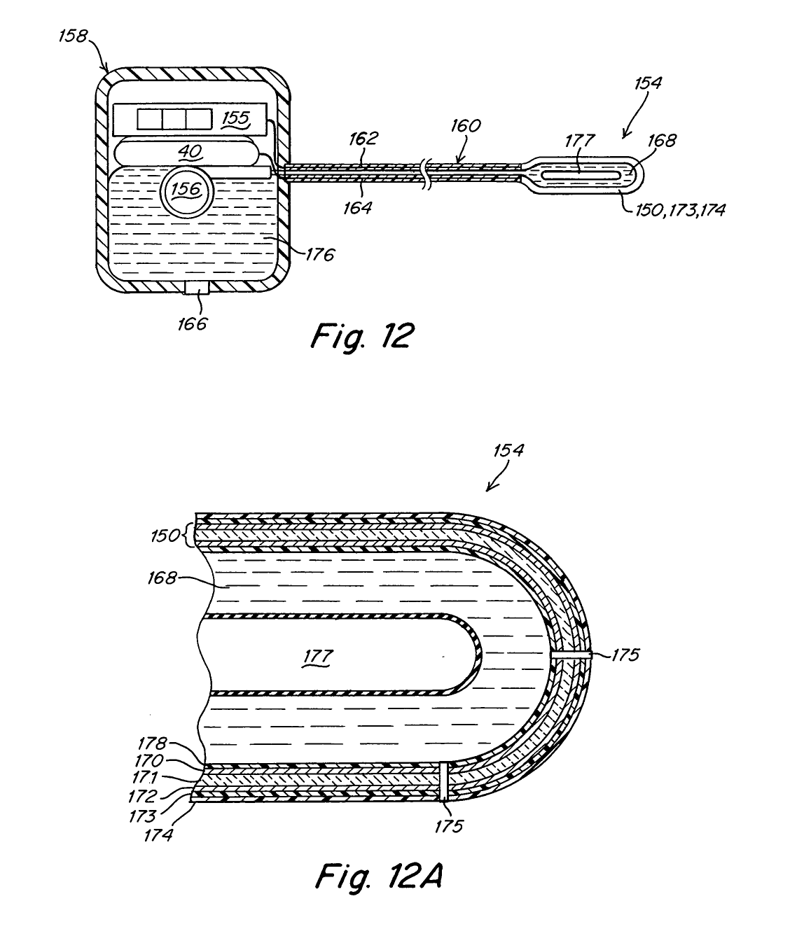

The control means may include a system for applying pressure to the reservoir contents, such as an infusion pump operatively coupled to a fluid port 48 in communication with the reservoir 36. As will be described in connection with the embodiments shown in FIGS. 4 and 12, a resilient bladder 50 may be provided to controllably pressurize, and in part define, the reservoir. The pressure source may be implanted in the patient, may be piggy-backed on another implanted device or may be disposed externally of the patient. In either case, the pressure source preferably is connected to the bladder through a lumen in an umbilical cord. By way of example, a variety of syringe pumps are available which are suitable for use in the invention. Manufacturers include Harvard Apparatus and Instech Instruments. Each manufactures a pump that will apply pressure to a standard 5 to 50 cc syringe in a controlled manner, thus allowing metered flow of the substance from the syringe to the device. Preferably the various functions of the system may be computer controlled. The control system for the device should include an arrangement for maintaining and varying the pressure applied to the reservoir for the prescribed duty cycle.

Any one or a combination of the controls may be used for switching the device between substance-delivering ‘on’ and delivery-stopping ‘off’ modes. This is a desirable safety feature which may be used in conjunction with or independent of other on/off control options. For example, switching off the transducer 38 would immediately stop ultrasonic wave generation or switching off the pressure would immediately terminate substance release.

The control systems should permit variable control and regulation of the treatment profile. Substance type, substance dosage profile and ultrasound parameters such as operation duty-cycle and wave intensity are examples of parameters that may be controlled during the course of treatment. Such controls enable the treatment regimen to be variably controlled as the patient's condition or response to treatment progresses.

The transducer 38 may be comprised of any suitable piezoelectric material such as those based on polymers, ceramics, and micromachined silicon wafers, as described by Van Lintell, et al., Sensors and Actuators (1988) 15(2):153-167. PZT is a presently preferred ceramic and should be fabricated to generate ultrasound at a frequency that will cause phonophoresis. A presently preferred range of frequencies is between about 20 KHz and 2 MHz. Among the materials of the polymeric type are included PVDF (polyvinylidene fluorides) and PVDF-TRFE (polyvinylidene fluoridetrifluoroethylene) as described by Chan, H. L. W., et al. (2000) IEE Transacts: On Dielectrics and Electrical Insulation, vol. 7(2) pp.204-207. Suitable ceramics include lead zirconate-titanate (PZT) with or without dopants, lead titanate (PT) and lead metaniobate (PMN). Also, for transducers employing planar structures, suitable materials may include lithium niobate, lead based single piezoelectric crystals, or magnetostrictive materials such as Terfenol.

The transducer 38 is powered by an energy module 40 that may be implanted as an integral part of or independent of the device, may be piggy-backed on another implanted device or may be external to the body. The control module 42, which may be internal or external the body or the device, controls operation of the power module 40, transducer 38 and operation of the system for pumping the substance. The power module 40 may comprise a battery or it may be any other operatively connected energy source. In some applications, it may be desirable for the energy module 40 to rely on an external power source, such as an induction based power transfer system by which an inductive field is applied externally of the patient to induce current in an implanted circuit and effect excitation of the piezoelectric transducer. Although power transfer through induction may result in reduced efficiency of energy transfer, it does not require opening of the skin and may, therefore, reduce the risk of infection.

The control module 42 controls signals to and from the transducer 38 and also may generate and amplify electrical signals for driving the transducer 38. Thus, the control module 42 preferably comprises a function generator 43, a signal amplifier 45, and a device logic controller 47. The function generator 43 comprises, for example, a programmable 0-15 MHz waveform generator. Signals from the function generator 43 such as a square wave or a sine wave, are amplified through the amplifier 45, such as a Class D amplifier, and applied to the transducer 38 which converts the electrical signal into the ultrasound energy.

The device logic controller 47 enables regulation of the signal from the function generator 43 and any associated amplification stages 45. The device logic controller 47 should also enable regulation of the duty cycle of the transducer 38, in either continuous operation or in burst mode, where in burst mode the duty cycle may range from less than 1% to more than 50%. Similarly, the device logic controller 47 regulates the number of bursts which may be varied from 1 to more than a 1,000. The device logic controller 47 preferably may provide a function for enabling or disabling device operation as well as modification of device operation-related parameters in real time. The amplitude of the voltage supplied to the piezoelectric layer material may be in the range of 600 to 3,000 volts peak-to-peak. The power is adjusted to achieve the desired substance uptake rate. Preferably the power is modulated over time so as to deliver physiologically acceptable concentrations of the substance.

The control module 42 also may control functions of other device components via the logic controller 47, as well as providing signal communication with a location external the body. For example, the control module 42 may further comprise a remote control option, permitting the clinician to monitor the status and operation of the device as well as to adjust the device operation-related parameters during treatment. The elements of the control module 42 may be contained on one or more suitable commercially available integrated circuit chips. The control module 42 may also include means to enable communication with a remote device outside the patient, as by wired or wireless telemetry means, for example, by Blue-Tooth communication protocol. A designated port 50 (FIG. 3) may be used for wired communication between the implanted device and an external location, or may be carried via an electrical conductor carried by the umbilical cord and coupled through the port 48 and the subcutaneous portal 30.

The ultrasound energy that is emitted from the device is at an energy level that will not substantially adversely affect the viability of the target tissue and cellular elements. Therefore, the characteristics of the emitted ultrasound energy are such that no substantial tissue necrosis or other irreversible effect will occur. Negative thermal ultrasound bio effects are generally avoided by controlling the intensity of the ultrasound The energy levels also may be controlled by operating the control module to appropriately vary the duty cycle and other control parameters. The present invention may be contrasted with conventional ultrasound techniques in which the ultrasound is either focused or adapted to generate elevated thermal levels and is within a frequency range outside that of the present invention.

The invention may be practiced in varying configurations. While in some embodiments it may be preferable to implant most or all of the components as a single integrated unit, other circumstances may call for implantation of some components while other components are either implanted elsewhere in the patient or are located externally of the patient and coupled to the implanted elements, by an umbilical cord. An umbilical cord may be provided with one or more operative channels between one or more access ports on the implanted device and a subcutaneous portal 30 for connection to the associated element external to the body. The cord may provide fluid communication to refill an implanted substance reservoir 36. Similarly, one or more electrical conductors may be provided to recharge the implanted battery 40, switch the transducer 38 ‘on/off’ via device logic controller 47, carry the excitation signal to the transducer 38 or carry data between the device and an external remote device.

FIG. 4 illustrates, somewhat diagrammatically, a spherical embodiment of an integrated phonophoresis substance delivery device capable of delivering the therapeutic substance and transmitting therapeutic ultrasound energy in an omnidirectional pattern to enhance substance uptake in the surrounding target area. A spherical embodiment is suitable particularly for treatment of brain tumors, as suggested in FIG. 2, or in other clinical situations where a pocket or cavity has been surgically formed and where the target is the tissue that surrounds and defines the cavity.

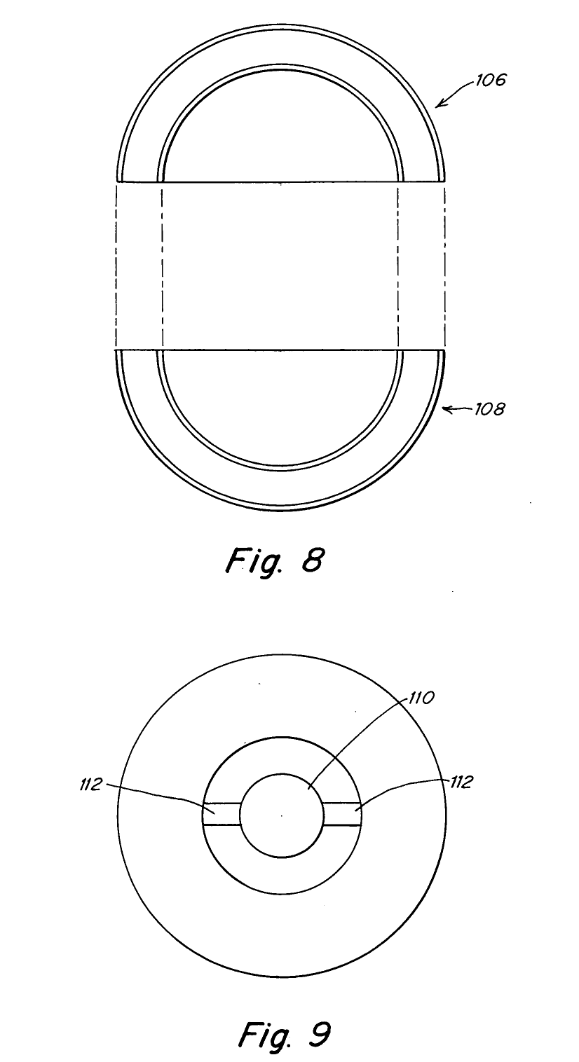

The spherical device may be formed in two separate hemispherical sections that are joined to form the sphere, as suggested in FIG. 8 and described in further detail below. When the embodiment of FIG. 4 is assembled it may be considered as a multilayered spherical shell, the innermost layer of which comprises a resilient bladder 50 defining a pressure chamber 52, arranged to apply pressure to the substance reservoir 36A to control substance delivery. The outer surface of the bladder 50 should be inert to the therapeutic substance and any fluid or fluent carriers by which the substance may be carried. In the spherical embodiment of FIG. 4, the outer surface of the bladder 50 defines the inner boundary of a spherical void that serves as the reservoir 36A adapted to contain the flowable therapeutic substance 54. The outer boundary of the spherical reservoir 36A is defined by another layer 56 that also is inert to the contents of the reservoir 36A and is electrically insulative. In this embodiment, the next outermost layer may comprise the piezoelectric material 58 of the ultrasound transducer in the form of a spherical shell having an inner spherical electrode 60 conductively attached to the inner surface of the piezoelectric material 58 and an outer spherical electrode 62 conductively attached to the outer surface of the piezoelectric material 58. The next outermost layer of the device may be an acoustic matching layer 64. The outermost layer 66 defines the substance emission surface and should be biocompatible. The biocompatible outermost layer 66 of the device isolates all of the components from body fluids to protect the device and the patient. The layer 66 may be formed, for example, from silicone, high density polyethylene (HDPE), or polycaprolactone (PCL). In this embodiment it may be desirable to incorporate an internal ultrasound absorbing member, such as an ultrasound absorbing sphere, as described more fully in connection with FIG. 9.

Therapeutic substance is delivered from the reservoir 36A to the target tissue through a plurality of valved pores 68 that provide fluid communication between the reservoir 36A and the exterior of the device. The pores 68 are formed through the composite spherical shell to communicate the reservoir 36 with the substance emission surface 44. The pores may be counterbored at each end. Although the pores 68 may be arranged in various patterns, the embodiment of FIG. 4 illustrates the pores as arranged in uniform circumferential spacing about the sphere. The number and distribution of pores 68 about the device should be selected to provide the desired pattern and quantity of therapeutic substance distribution.

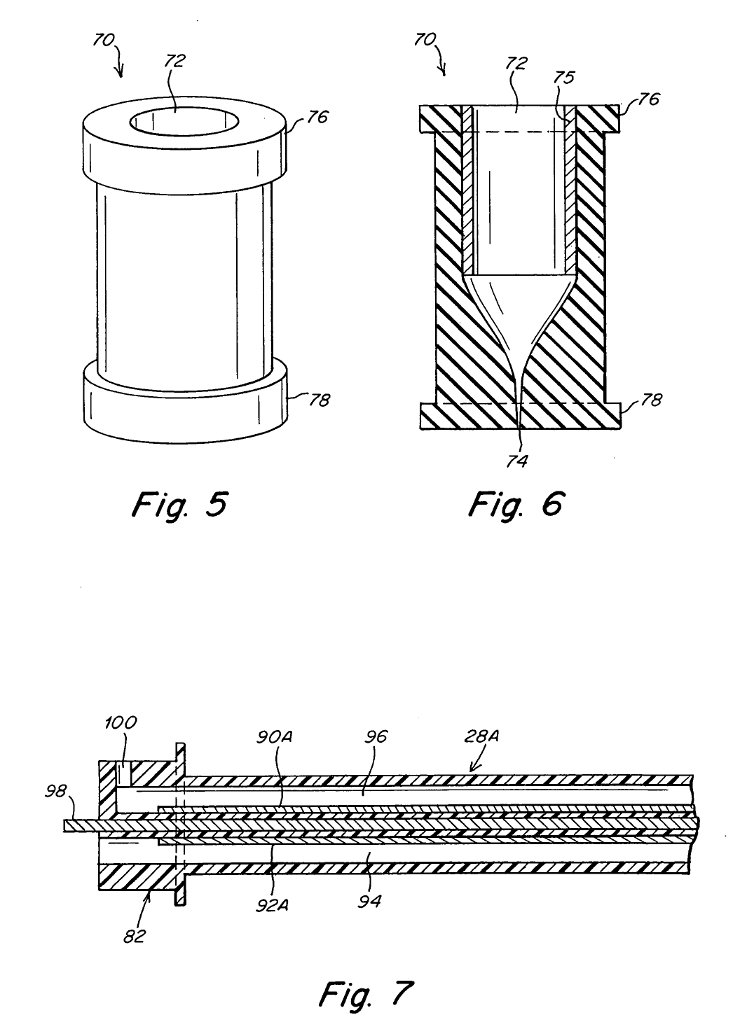

FIGS. 5 and 6 illustrate, somewhat diagrammatically, a one way valve 70 that may be disposed in the pores 68 in order to control emission of the therapeutic substance. Each valve is formed from a resilient material, such as a curable silicone polymer, and includes an inlet end 72 and an outlet end 74. The valve 70 may be provided with retention flanges 76, 78 to facilitate secure attachment of the valves 70 within the pores 68. The inlet end 72 of the valve may define a larger flow passageway that at the outlet end 74 and may be reinforced by a stiff tube 75. The outlet, as shown, may be formed to taper to a narrow constriction sufficient to prevent fluid or fluent therapeutic material from passing out of the device in the absence of a predetermined threshold pressure applied to the inlet side of the valve. The valve is formed from a resilient material such that when the threshold pressure is reached, the valve outlet 74 will expand under the influence of that pressure to enable therapeutic material to flow from the reservoir, through the valve 70 and into the surrounding cavity or pocket in the target tissue.

The one way valve 70 may be formed, for example, by filling the pores 68 of the shell with curable silicone material and then, while the silicone is still fluent and formable, inserting a slender, wire-like mandril through the silicone and permitting the silicone to cure with the mandril in place. The ends of the pores 68 are counterbored to receive the fluent silicone and form the flanges 76, 78. Upon curing, the mandril may be withdrawn leaving the desired internally contoured passage through the valve 70. By way of example, the pores may have a diameter of the order of 1 mm with the channel size, formed by the wire, being in the range of about 0.001 to about 0.010 inches diameter.

The device also may include a connector 80 that extends radially through the spherical shell to provide communication with the internal components of the device by which the device may be operated and controlled. The connector 80 is connectible to another connector 82 that, in turn, is connected to an umbilical cord 28A.

As shown in FIG. 4 embodiment, the control module 42 and the power module 40 may be contained and mounted within the pressure chamber 52. The power module may comprise a battery 40 operatively connected to the control module 42. As described above, the control module 42 may include a function generator 43, a signal amplifier 45, and a device logic controller 47 for controlling operation of the device. The control module 42 may be electrically coupled to inner and outer electrode layers 60, 62 by insulated electrical conductors 90, 92 that also may be used to recharge the battery power source 40 through the control module. The battery 40 and the control module 42 are contained within the bladder and should be sealed to isolate the electrical circuitry from contamination by pressurizing fluid or bodily fluids. As may be seen, the connector 80 also provides routing for the conductors 90, 92 permitting electrical connection between the inner and outer electrode layers 60, 62 and the control module 42, while permitting the bladder 50 to be joined and sealed to the connector 80. Another wiring arrangement 104 provides for data communication with the control module 42. The wiring arrangement 104 is associated with contacts (not shown) in the connector 80 that are connectible with mating electrical contacts associated with wiring 98 that extends through the umbilical cord 28A to the connector 82. The umbilical cord (FIG. 7) also includes power conductors 90A, 92A connectible to the power lines 90, 92 as well as the wiring 98 for signal transfer. The conductors 90A, 92A may be associated with contacts 91 on the connector 82 that are mateable, through connector 80, to conductors 90, 92.

In the embodiment of FIG. 4, means, such as a pump, are provided to pressurize the chamber 52 to regulate the delivery of therapeutic substance contained in the reservoir 36A. The pump is connectable to the chamber by the cord 28A. As shown in FIG. 7 the umbilical cord 28A may be detachably connectable to the spherical device by mating the connectors 80, 82. The other end of the umbilical cord 28A may be connected to the portal 30 which may be imbedded subcutaneously or be maintained externally of the body. The connector 80 includes electrical conductors adapted to perform the functions described above.

The umbilical cord 28A includes a lumen 94 for communicating fluid under pressure with the inflation chamber 52 of the bladder 50 through a channel (not shown) in connector 80. The pump may be implanted in the patient or may be located externally. The pressurized fluid may be a biologically compatible solution such, for example, as normal saline, alcoholic saline or ringers lactate. Upon pressurization, the bladder 50 expands, thereby applying pressure to the therapeutic substance in the reservoir 36A to force it through the valves 70. The umbilical cord also may include a delivery channel 96 by which the reservoir 36A may be filled or replenished with therapeutic substance. The channel 96 communicates with a radial passageway 100 formed in the connector 82. When the connectors 80, 82 are coupled, the passageway 100 in connector 82 registers with another radial passageway 102 in connector 80 that opens into the reservoir 36A. The connector 80 also may include a lead 104 by which a conductor 98 carried by the umbilical cord 28A can be electrically connected within the connector 80, to the control module 42.

FIGS. 8 and 9 illustrate, diagrammatically, the manner in which the spherical device of FIG. 4 may be made. As seen in FIG. 8, the device is made in two hemispherical shell-like halves 106, 108. Each hemispherical portion is built up in layers. By forming the device in hemispherical sections, the inner and outer surfaces will be accessible before the device is assembled, facilitating placement of electrodes, forming of the pores 68 and valves, and other internal assembly operations. When the components for each of the hemispherical portions 106, 108 have been completed and secured within the shell, the two halves are joined to each other. For the embodiment illustrated in FIGS. 4 and 8, the hemispherical piezoelectric transducers are formed as hemispherical shells. When the piezoelectric material comprises a ceramic, as in the preferred embodiment, the shells 58 may be machined from a selected solid ceramic block to specifications and dimensions appropriate for the intended device. The interior and exterior surfaces of the hemispherical shell then are metallized, to form the inner and outer electrodes 60, 62, respectively, either by sputtering, by electroless plating or by conductive ink, using techniques well known to those skilled in the art of piezoelectric fabrication. The transducer hemispheres then are subjected to a high voltage in a hot oil bath to affect polarization. The inner surface of the inner electrode 60 is covered with a layer of electrically insulative material that also defines the outer surface of the spherical shell-shaped reservoir 36A. The innermost surface of the reservoir will be defined by the outer surface of the flexible, resilient bladder 50 which can be fabricated separately and placed inside the shells when the hemispheres are assembled. The outer surface of the outer spherical shell electrode 62 may be covered with a layer 64 of acoustic matching material. The acoustic matching layer 64 may be biocompatible or may be covered by another outermost layer 66 of a biocompatible material.

During the manufacture of the hemispherical shells, after the electrodes have been formed on the ceramic, a plurality of radially oriented pores 68 are formed through the hemispherical shells, as by drilling with diamond tooling. The pores may be arranged to be substantially uniformly spaced about the spherical surface of the device to facilitate uniform distributions of therapeutic substance. By way of example, for spherical device having a diameter of the order of 2 cm, approximately eight to twelve pores can be placed along an equatorial line with the spacing between adjacent pores 68 being of the order of 5 mm. Each pore may be of the order of 1 to 1.5 mm diameter. The pores extend radially to provide fluid communication between the reservoir 36A and the outer surface 44 of the device. Pressure responsive, one-way valves, are located within the pores 68 to enable therapeutic fluid to flow from the reservoir to the surface 44 when the reservoir is pressurized. The one-way valves may be formed as described above in connection with FIGS. 5 and 6.

FIG. 9 illustrates, diagrammatically, the manner in which the hemispherical sections, when assembled, may include an internal ultrasound absorbing member, such as a smaller silicone sphere 110. The sphere 110 may be supported, as by silicone standoffs 112 formed before final assembly of the device.

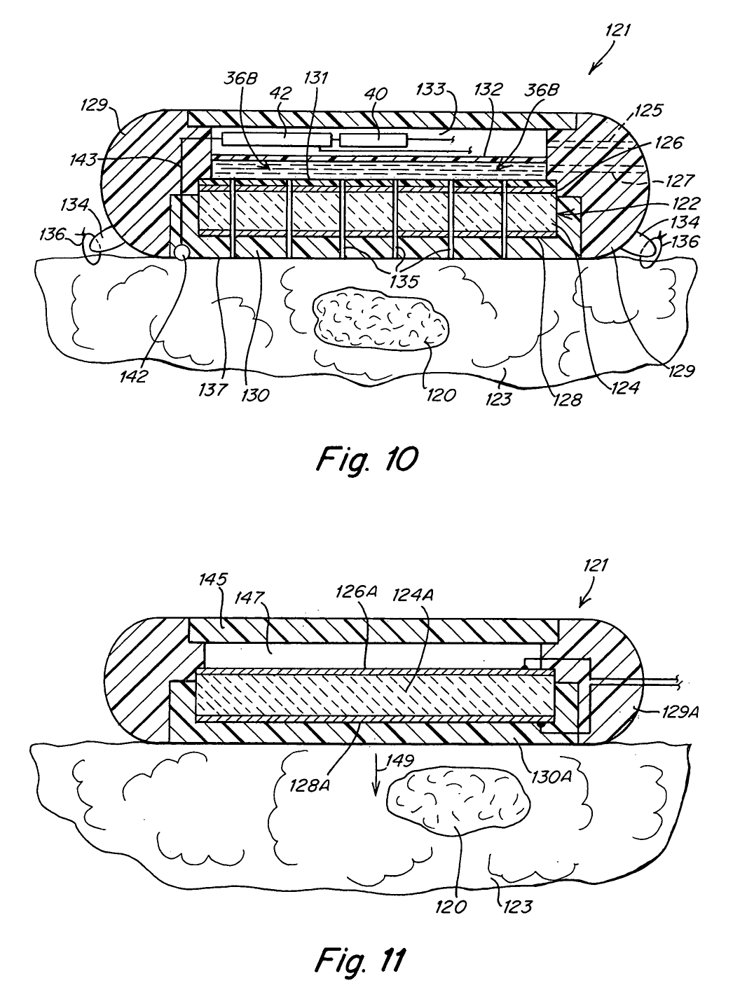

The invention may be practiced with devices having configurations other than spherical. The form of the device may be dictated, in part, by the requirements and characteristics of the particular implantation site. FIG. 10 illustrates, somewhat diagrammatically in cross section, one such alternative configuration in which the target tissue 120 has dimensions, a shape or is positioned such that a non-spherical ultrasound field would be more appropriate. In this embodiment, the device 121 has a transducer 122 that is flat, having a piezoelectric layer 124 with conductive electrode layers 126, 128 formed on opposite sides of the piezoelectric layer 124. The device may include an annular frame 129 that defines the periphery of the device and provides support for other internally contained components. An ultrasound matching layer 130 is provided on the ultrasound emission face of the transducer 122. The matching layer is selected based on the characteristics of the target tissue 120. Generally, for soft tissue, such as suggested at 120 or 123, one type of matching layer may suffice. For hard tissue (bone) another type of matching layer may be appropriate. The innermost electrode layer 126 should be covered with a layer 131 of material that is inert to the selected therapeutic substance. The inert layer 131 also may define one surface of a reservoir 36B. There may be instances in which it would be desirable to block rearward transmission, for example, to protect the substance molecules from long exposures to ultrasound energy. In such cases layer 131 can be fabricated to form an anti-transmission layer to block such exposure. The reservoir may be enclosed by a reservoir wall 132. The back side of the piezoelectric ceramic also may comprise an anti-transmission layer (not shown) to reflect most of the ultrasound energy radiated in the rearward direction to the forward direction, thus avoiding potentially destructive interfering acoustic reflections from components placed behind the ceramic. The device may include a chamber 133 adapted to house a power source 40 and a control module 42 as described above in connection with the embodiment shown in FIG. 4. Passageways, shown diagrammatically at 125 and 127 may be formed through the frame 129 for passage of electrical wires and for enabling refilling of the reservoir from an implanted or an external source. Suitable electrical conductors may extend through the housing as through the ring 129, to couple the devise with computer controls.

A pump (not shown) is provided, either implanted or located externally, to increase the pressure of the fluid or fluent therapeutic substance to cause it to flow from the reservoir through valved pores 135 that extend between the reservoir 36B and the substrate emission surface 137 of the device. The device also may be provided with an attachment ring 134 extending about the periphery of the device by which the device can be secured to tissue, as by sutures 136, stapling or other means. Alternatively, the device may be secured in place with biocompatible adhesive. The entire device may be encapsulated within a layer of a suitable biocompatible material, such as, for example, silicone or high density polyethylene. The device also may include a temperature sensor 142 coupled to the control module 42 as by wire 143. The sensor 142 may trigger selected functions, such as shut-off power if the temperature exceeds a predetermined value. The device logic controller may restart the device after a cool-down period.

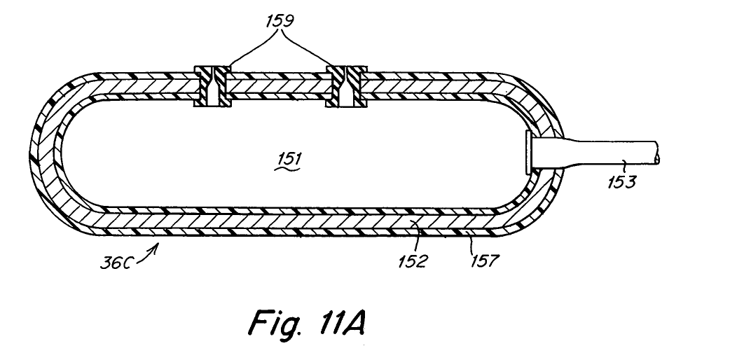

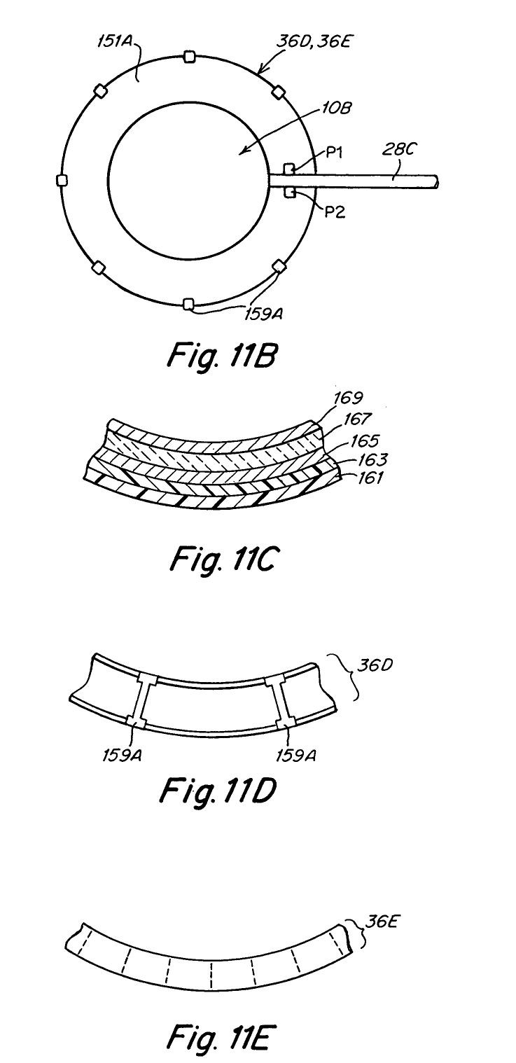

FIG. 11 illustrates a device similar in construction to that of FIG. 10 except that it functions as an ultrasound-only device that does not include a reservoir and is configured only to emit ultrasound energy from one side of the device indicated by arrow 149. The device may include a frame, such as a ring-like frame 129A, that houses an ultrasound transducer comprising a piezoelectric ceramic element 124A with conductive electrodes, 126A, 128A. A back cover 145 is secured to the rear face of the frame 129A in spaced relation to the rear face of the transducer to define an air space 147. The rearward transmission of ultrasound is effectively blocked by the impedance mismatch between the ceramic and the air 147, with a transmission coefficient of less than −70 dB. The front face of the device includes a matching layer 130A, formed from a material and of a thickness to provide the desired acoustic coupling Wiring is connected to the electrodes. The entire device, including projecting wires is encapsulated in a layer of suitably selected biocompatible material.

FIG. 1A illustrates a separately implantable reservoir that may be used, for example, with a separately implantable ultrasonic transducer, such as those illustrated in FIGS. 1 and 11. The reservoir, indicated generally at 36C is adapted to receive and deliver a flowable therapeutic substance in a reservoir chamber 151. The chamber 151 may be filled with therapeutic substance delivered through an umbilical cord 153 that communicates with the chamber 151 and is connected to a controllable source of pressure. The wall that defines the reservoir includes a structural wall 153 having an inner layer formed from a material inert to the substance contained in the chamber 151, and an outer biocompatible layer 157. Flowable material may be emitted from the reservoir 36C by outlet ports 159 formed through the reservoir wall. The outlet ports 159 may have the configuration described above in connection with the embodiment shown in FIGS. 5 and 6.

FIGS. 11B-11E illustrate, diagrammatically, an embodiment in which the ultrasound transducer 10B is contained within the reservoir 36D. In this embodiment, shown in a spherical configuration, the transducer 10B may be considered to include a shell having an outer biocompatible layer 161 that defines a surface of the internal volume 151A of the reservoir 36D, a next innermost matching layer 163 an outer conductive layer 165, the PZT layer 167 and an inner conductive layer 169. The conductive layers 165, 169 are connected electrically to the electrical components of the device (not shown in this embodiment). The electronic components may be located internally of the transducer 10B or may be located externally of the device and connected by an umbilical cord 28C. FIG. 11D illustrates a cross-sectional portion of the wall of the reservoir 36D. The wall may be formed from a biocompatible material that may be flexible or rigid. Alternately, it may be formed from a material that is not itself biocompatible and is provided with inner and outer biocompatible layers. Pores are formed through the wall of the reservoir 36D and are provided with one way valves 159A. The valves 159A may have the same construction described above in connection with the previous embodiments. FIG. 11E illustrates another configuration for the reservoir wall 36E, in which the wall may comprise a thin polymer membrane having a multitude of micropores dimensioned to allow weeping of the flowable therapeutic agent through the reservoir wall under the influence of a pressure differential across the wall. The microporous surface may be formed in a variety of techniques, such as, for example only, irradiation of the polymer membrane.

The umbilical cord 28C may include lumens to communicate with the reservoir chamber 151A. One or more ports P1, P2 may be provided to communicate corresponding lumens in the umbilical cord 28C with the reservoir chamber 151A. One of the ports may be associated with an umbilical cord lumen connected to a controllable pressure source. The other port may be provided to enable introduction or withdrawal of fluid from the reservoir chamber 151A. In this embodiment, the ultrasound is radiated outwardly, through the reservoir chamber 151 and reservoir wall 36D to apply, controllably, ultrasound to a therapeutic substance delivered to the surrounding tissue through outlets 159A.

FIG. 12 shows, diagrammatically, another embodiment that may be usable in confined settings where the region about the target is able only to receive a very small, compact device. In this embodiment a compact ultrasound transducer 150 and a small reservoir 168 are provided in a relatively small module 154 adapted to be implanted in the region of the target. Other components of the system, such as a control module 155 including a function generator, signal amplifier and logic controller, as well as a power source 40 and a pressurizing means, such as a pump 156, can be contained in a separate housing 158 which may be implanted at an anatomically remote location or may be located externally of the patient. The internal components of the housing 158 and the distal module 154 are connected by an umbilical cord 160 carrying conductive wires 162, 164 that couple the electrodes of the ultrasound transducer 150 of the module 154 with the components contained in the housing 158. The housing also may include a replenishment reservoir 165 to provide sufficient therapeutic substance to the relatively small reservoir 168 in the distal module 154. An access port 166 may be formed in the housing and may be connected via an additional umbilical cord (not shown) to an access portal (not shown) through which reservoir may be replenished, battery recharged or control module be externally controlled.

FIG. 12A illustrates, diagrammatically, and in enlarged detail the configuration of the distal module 154. The module includes an inner resilient bladder 167, a reservoir 168 for containing flowable therapeutic substance and an elongate shell that may have a smoothly curved forward end. The module 154 may be generally cylindrical. The shell includes an inner biocompatible layer 169 formed from a material that also is inert to the substance contained in the reservoir 168. The next outermost layer comprises the inner conductor 170 for the piezoelectric transducer, then the shaped piezoelectric layer 171 and then the outer electrode layer 172. An acoustic matching layer 173 covers the outer electrode layer and an outer biocompatible layer 174 defines the outer surface of the device. A plurality of pores 175, which may have one-way valves are formed through the shell to communicate the reservoir 168 with the tissue surrounding the module 154.

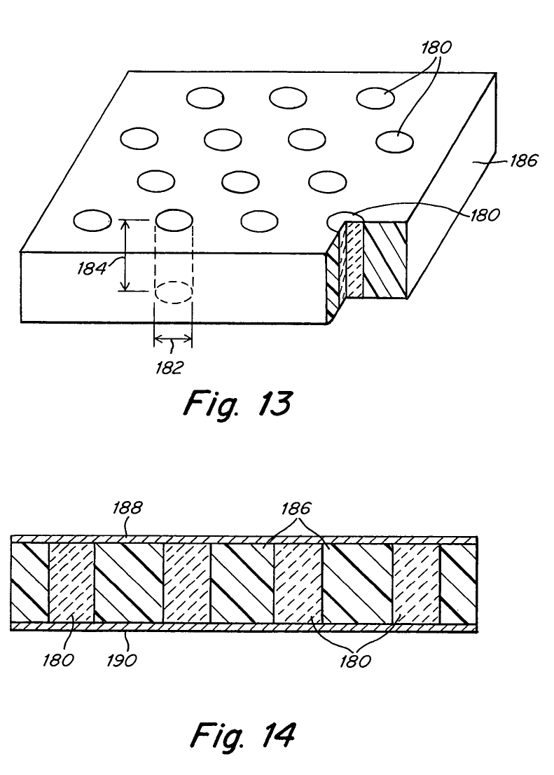

Devices embodying the various aspects of the invention also may be fabricated from piezo composite materials in which piezoelectric segments are embedded within a polymer matrix. Piezoelectric ceramics typically exhibit vibration modes along a primary direction, corresponding to the thickness of the ceramic, as well as strong lateral vibrations. It is desirable to maximize the proportion of vibration that is directed along the thickness direction. By cutting the ceramic element such that its widthwise dimension is not substantially more than about two-thirds that of the thickness dimension, the extent of ultrasonic vibration in a lateral direction is sufficiently disrupted to cause a greater energy transfer in the thickness direction as well as an overall greater efficiency of energy transfer from the electrical signal through the transducer and into the subject tissue. FIG. 13 illustrates, diagrammatically, a transducer in which a plurality of individual “posts” 180 of ceramic piezoelectric material are formed in which each post 180 has a ratio of lateral dimension 182 to thickness dimension 184 that is relatively small, preferably less than two-thirds. The posts 180 are embedded in a polymeric matrix 186. The posts may have circular, square or any other cross section. Ground and signal electrodes 188, 190 are placed on the front and back surfaces of the composite as suggested in FIG. 14. The polymer may be selected from any of a variety of materials, such as polyurethanes, epoxies and the like. Due to the overall reduced mass and, consequently, acoustic impedance of such a composite arrangement, it may be possible to avoid the requirement of an impedance matching layer at the emissions surface of the device. The polymer matrix, however, should be selected carefully to provide adequate mechanical strength, light weight and brittleness for minimum heat buildup in the composite.

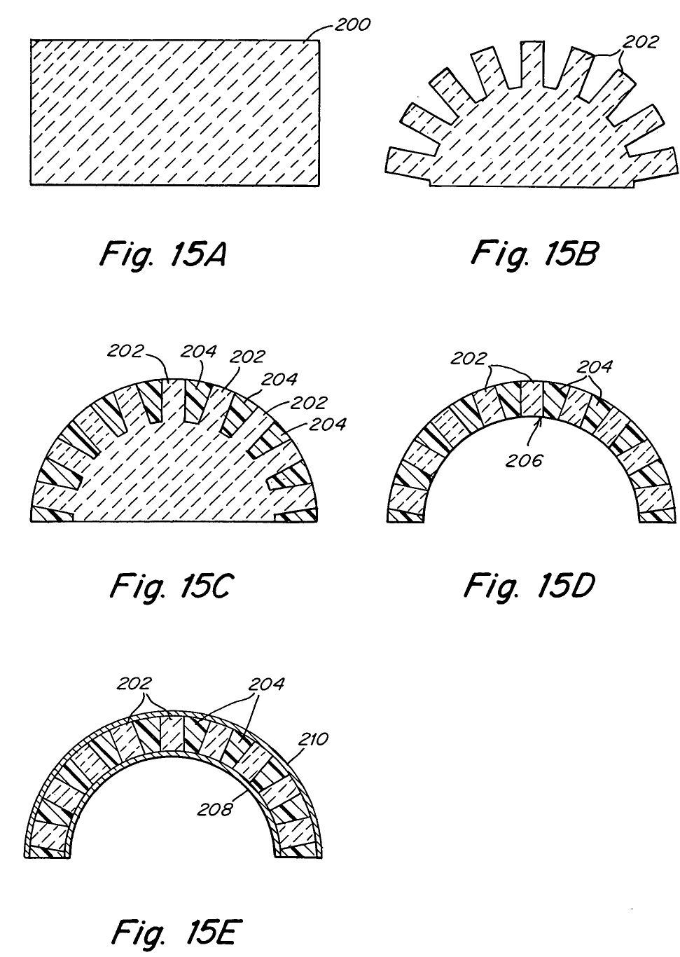

FIGS. 15A-15E illustrate, diagrammatically, a series of steps for making a spherical transducer in a piezo composite configuration. FIG. 15A illustrates a block 200 of piezoelectric ceramic. FIG. 15B illustrates the block after it has been machined or ground to form a convex hemispherical surface, as with diamond cutting tools. The hemispherical ceramic is then mounted on a rotary table with an elevation adjustment and, using a thin walled diamond core drill, ceramic posts 202 are cut in the hemispherical surface, deeper than the actual desired thickness of the finished piece. FIG. 15C illustrates the cut-away volume as being filled with the polymer matrix material 204. This may be achieved by inverting the hemispherical element into a concave hemispherical bowl (not shown) filled with the polymer material in a fluent form. Trapped air should be evacuated. After the polymer matrix material has cured fully, an external convex hemispherical surface may again need to be cut onto the assembly, due to slight overflow of the polymer matrix material. FIG. 15D illustrates the device after an internal concave surface 206 has been ground into the composite structure. The resulting hemispherical shell includes a plurality of ceramic posts embedded in the polymer matrix. The inner and outer surfaces of the hemispherical shell then may be metalized, either by sputtering or by conductive ink to form electrodes 208, 210. Following metalization, the device is polarized.

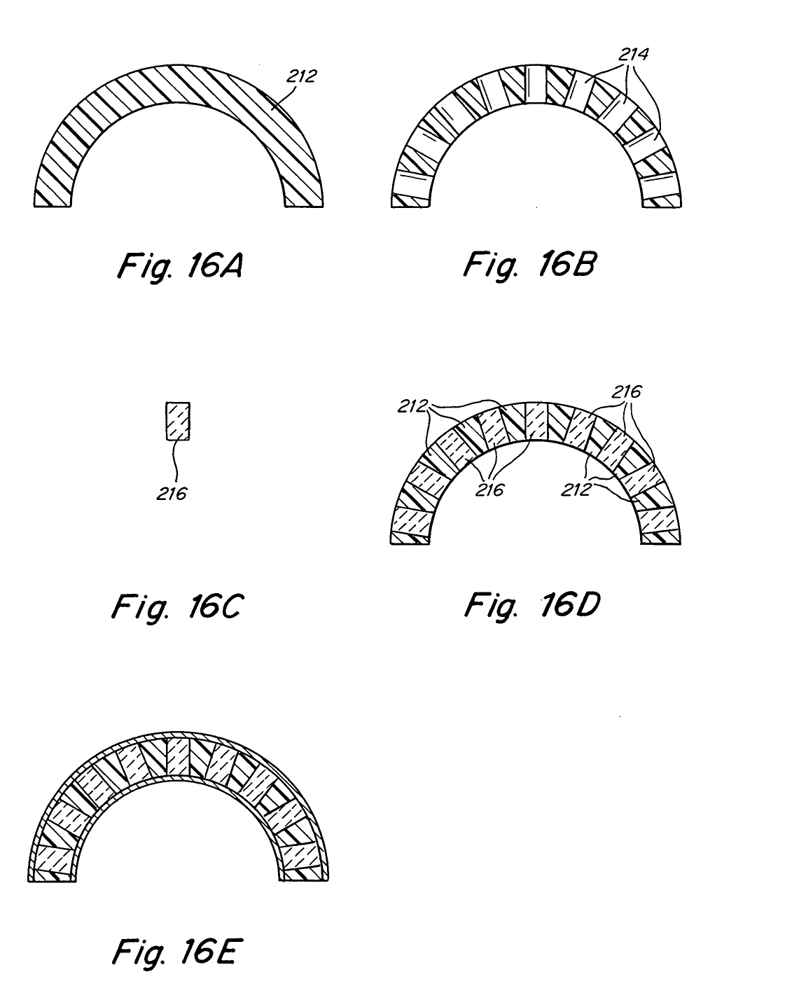

FIGS. 16A through 16E illustrate a second approach to fabrication of a hemispherical shell piezo composite. FIG. 16A illustrates the starting material in the form a polymeric hemispherical shell 212 which may be fabricated by machining a solid block of material or by cast in a shape between appropriate mold surfaces. The shell is then mounted on a rotary table with elevation control and, as shown in FIG. 16B, a cluster of holes 214 is drilled to a diameter adapted to receive the piezoelectric posts, shown in FIG. 16C, which will be inserted into the holes. The posts 216 may be cut using diamond core drills from plates of piezoelectric ceramic with the inner and outer surfaces of the posts being metalized and polarized. As shown in FIG. 16D, the posts are slid into the holes in the hemispherical shells and may be held in place with a minute amount of epoxy or cyanoacrylate adhesive. An internal convex negative might be placed into the concave side of the hemisphere to assure a proper depth for all of the ceramic inserts. Following appropriate cleaning of the internal and external surfaces, common electrodes connecting all of the piezoelectric posts can be applied as shown in FIG. 16E. Alternately, if a quarter wave impedance matching layer is applied over the composite structure, individual post electrodes first may be connected by the soldering of a small diameter lead wire before the device is covered with a matching layer.

The devices may be constructed and controlled to provide some limited range of emitted ultrasound frequencies. By the appropriate implementation of one or more front surface impedance matching layers, the bandwidth of the device may be broadened substantially. Techniques for creating one or more impedance matching layers are well known to those skilled in the art. Combinations may be selected to allow up to 50 to 100% bandwidth about the center frequency. Thus, the signal generator may be adjusted to cause device operation at any frequency within the achieved band width.

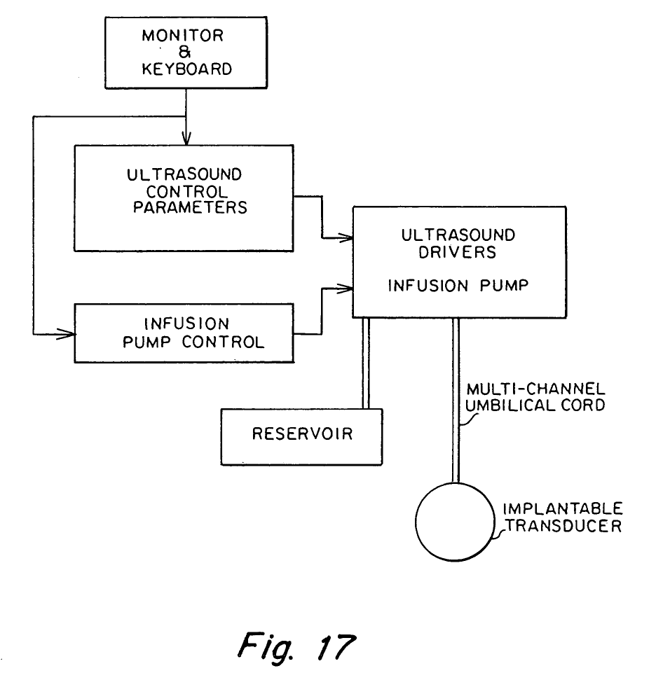

FIG. 17 illustrates a block diagram of the relationship of the control elements as they might be configured to provide the clinician with keyboard real-time control of the ultrasound and infusion delivery operation. The arrangement includes the substance reservoir and the ultrasound transducer. Although the substance reservoir is shown in FIG. 17 as separate from the transducer, they may be combined in a unitary configuration, as described, for example, in connection with FIG. 4. The ultrasound transducer and reservoir are operationally coupled to the ultrasound drivers and a pump for effecting delivery of the substance from the reservoir to the transducer through a multi-channel umbilical cord adapted to have electrical and fluid transmitting channels. The control electronics are adapted to operate the ultrasound drivers to control the intensity, duty cycle, on/off protocols as well as other parameters associated with operation of the ultrasound transducers. The ultrasound control parameters can be varied by being coupled with a computer and keyboard. The electronics for controlling the pump also are coupled with the computer to enable the clinician to control its operation as well. By enabling control, in real-time, of the ultrasound and pumping operation, the clinician can redefine and program a treatment profile. For example, if it is desired to change a currently released drug for another, the substance reservoir may be filled with the replacement drug. A new drug release profile can be directed to the infusion pump controls and a new ultrasound treatment profile can be applied to the ultrasound control parameters.

It should be understood that the foregoing description of the invention and its various aspects is intended merely to be illustrative thereof and that modifications and equivalents may be devised while embodying the principles of the invention.

Although the following claims represent what we desire to claim as of the filing date of this application, it should be understood that further claims may be added and that the scope of the claims may be broadened as the prosecution of this application, or any application that relies for priority on this application, progresses.

We claim:

1. An ultrasound device for increasing the uptake of a locally delivered therapeutic substance to target tissue internally of a mammalian body comprising:
a surgically implantable ultrasound transducer configured to emit ultrasound energy at levels at which no substantial tissue necrosis occurs and in the frequency range of about 20 KHz to about 2 MHz to promote phonophoresis within tissue immediately adjacent the transducer;
the transducer being encapsulated in a biocompatible layer fully enclosing all external surfaces of the transducer;
control means for operating the transducer;
a power source operatively associated with the transducer for exciting the transducer to generate the ultrasound energy.
a surgically implantable source of therapeutic substance associated with the transducer, the source being configured to enable delivery of the substance directly to the immediate region of the target tissue and within the ultrasonic field of the ultrasound transducer whereby the ultrasound transducer can be operated to enhance the uptake of the therapeutic substance by the target tissue;
the source of therapeutic substance comprising microspheres containing the substance and being responsive to ultrasonic energy to release the substance from the microspheres.

2. An ultrasound device for increasing the uptake of a locally delivered therapeutic substance to target tissue internally of a mammalian body comprising:
a surgically implantable ultrasound transducer configured to emit ultrasound energy at levels at which no substantial tissue necrosis occurs and in the frequency range of about 20 KHz to about 2 MHz to promote phonophoresis within tissue immediately adjacent the transducer;
the transducer being encapsulated in a biocompatible layer fully enclosing all external surfaces of the transducer;
control means for operating the transducer;
a power source operatively associated with the transducer for exciting the transducer to generate the ultrasound energy.
a surgically implantable source of therapeutic substance associated with the transducer, the source being configured to enable delivery of the substance directly to the immediate region of the target tissue and within the ultrasonic field of the ultrasound transducer whereby the ultrasound transducer can be operated to enhance the uptake of the therapeutic substance by the target tissue;
the source comprising therapeutic substance contained in biodegradable polymer matrix.

3. An ultrasound device for increasing the uptake of a locally delivered therapeutic substance to target tissue internally of a mammalian body comprising:
a surgically implantable ultrasound transducer configured to emit ultrasound energy at levels at which no substantial tissue necrosis occurs and in the frequency range of about 20 KHz to about 2 MHz to promote phonophoresis within tissue immediately adjacent the transducer;
the transducer being encapsulated in a biocompatible layer fully enclosing all external surfaces of the transducer;
control means for operating the transducer;
a power source operatively associated with the transducer for exciting the transducer to generate the ultrasound energy.
a surgically implantable source of therapeutic substance associated with the transducer, the source being configured to enable delivery of the substance directly to the immediate region of the target tissue and within the ultrasonic field of the ultrasound transducer whereby the ultrasound transducer can be operated to enhance the uptake of the therapeutic substance by the target tissue;
wherein the substance is flowable at body temperature and wherein the source comprises a reservoir adapted to contain the flowable substance.

4. A device defined in claim 3 further comprising a pressure responsive valve having an inlet in communication with the reservoir and an outlet at the exterior of the device, the valve being operable in response to an increased pressure applied to the reservoir to cause the valve to open and enable the substance to flow out of the device.

5. A device as defined in claim 4 further comprising a pressure source operatively associated with the reservoir for pressurizing the reservoir.

6. A device defined in claim 5 wherein the pressure source is implantable and is operatively connected to the reservoir by an umbilical cord.

7. A device defined in claim 6 wherein the power source is implantable within the patient.

8. A device defined in claim 6 wherein the power source is adapted to be located externally of the body and is electrically connected to the transducer by an umbilical cord.

9. An ultrasound device for increasing the uptake of a locally delivered therapeutic substance to target tissue internally of a mammalian body comprising:
a surgically implantable ultrasound transducer configured to emit ultrasound energy at levels at which no substantial tissue necrosis occurs and in the frequency range of about 20 KHz to about 2 MHz to promote phonophoresis within tissue immediately adjacent the transducer;
the transducer being encapsulated in a biocompatible layer fully enclosing all external surfaces of the transducer;
control means for operating the transducer;
a power source operatively associated with the transducer for exciting the transducer to generate the ultrasound energy.
a surgically implantable source of therapeutic substance associated with the transducer, the source being configured to enable delivery of the substance directly to the immediate region of the target tissue and within the ultrasonic field of the ultrasound transducer whereby the ultrasound transducer can be operated to enhance the uptake of the therapeutic substance by the target tissue;
the source of therapeutic substance comprising microspheres containing the substance and being responsive to ultrasonic energy to release the substance from the microspheres;
the transducer and the power source being integrally coupled to and housed within an individually implantable device.

10. A device defined in claim 1 further comprising:
the transducer being adapted to emit ultrasound in a defined directional pattern;
a reservoir of flowable therapeutic substance located relative to the transducer to be substantially out of the pattern of the emitted ultrasound;
a passage through the transducer to enable the therapeutic substance to flow from the reservoir to an outlet port;
the outlet port being located to enable placement of therapeutic substance within the field of the directed ultrasound.

11. A device defined in claim 10 further comprising a pressure responsive valve disposed within the outlet port.

12. A device defined in claim 3 wherein the device is in the form of a multilayered spherical shell having a plurality of generally spherical layers and comprising:
a first, inner expandable layer defining an internal pressure chamber and having an outer surface defining a boundary of the reservoir;
a second layer outside of the first layer and defining another boundary of the reservoir, the second layer being spaced from the first layer to define the reservoir;
a third layer disposed outside of the second layer and comprising the ultrasound transducer;
a fourth layer outside of the third layer formed from an electrically insulative material; and
a plurality of pores formed through the multilayered shell, the pores including an inlet end in communication with the reservoir layer and an outlet end extending through the outermost layer to enable therapeutic substance to flow from the reservoir through the passageway to a location externally of the device where it may be exposed to tissue.

13. A device as defined in claim 12 further comprising means for pressurizing the chamber to force therapeutic substance from the reservoir through the passageways.

14. A device as defined in claim 12 further comprising an electronic control module disposed within the pressure chamber.

15. A device as defined in claim 14 wherein the control module comprises a function generator adapted to generate a signal for exciting the ultrasound transducer;
a signal amplifier coupled with the function generator and the transducer to enable variation in the amplitude of the generated signal; and
a device logic controller for controlling operation of electrical and substance delivery functions of the device.

16. A device defined in claim 15 further comprising:
a first connector extending through the shell to enable communication from a location external of the device with at least one of the pressure chamber and layers of the shell; and;
an umbilical cord having a second connector mateable with the first connector and having at least one of a lumen for applying fluid pressure to the pressure chamber and a conductive path to establish electrical connection with internal electrical components of the device.

17. A device as defined in claim 16 further comprising a lumen for replenishment of the reservoir with therapeutic substance.

18. A device as defined in claim 3 having a non-spherical shape comprising:
a non-spherical housing having front and back faces and defining an enclosed interior;
the reservoir being within the housing;
the ultrasound transducer being disposed between the reservoir and the front face of the housing; and
at least one passageway extending from the reservoir through the transducer to the front face of the housing to enable a flowable therapeutic substance to flow from the reservoir and out of the front face of the housing.

19. A device defined in claim 18 further comprising:
a valve located within the passageway for allowing flow substantially only in frontward direction.

20. A device as defined in claim 18 further comprising:
the back surface of the piezoelectric ceramic including an anti-transmissive layer to reduce propagation of ultrasound energy in the rearward direction.

21. A device defined in claim 20 wherein at least a portion of the control means is disposed within the space between the reservoir and the backing layer.

22. A device as defined in claim 20 wherein the power source is disposed in the space between the reservoir and the backing layer.

23. A device as defined in claim 22 further comprising the front face including an ultrasound matching layer.

24. A device defined in claim 3 wherein the ultrasound transducer comprises:
a plurality of ultrasound transducer elements contained, in spaced relation, in a polymeric matrix and defining an array of ultrasound emission surfaces, each transducer element having a thickness dimension terminating at end surfaces and a transverse dimension perpendicular to the thickness dimension;
the thickness direction being oriented along the primary direction of propagation of ultrasound waves;
a first electrode at one end face and a second electrode at the opposite end face;
the first electrodes of the plurality of transducer elements being electrically connected to each other; and
the second electrodes of the plurality of transducer elements being electrically connected to each other;
whereby all of the plurality of transducer elements may be excited in unison.

25. A device as defined in claim 24 wherein the ratio of the transverse dimension to the thickness dimension is not substantially greater than about 2:3.

26. A device as defined in claim 24 wherein the thickness dimension of each of the plurality of transducer elements is greater than the transverse dimension.

27. An ultrasound device for increasing the uptake of a locally delivered therapeutic substance to target tissue internally of a mammalian body comprising:
an ultrasound transducer configured to emit ultrasound energy at levels at which no substantial tissue necrosis occurs and in the frequency range of about 20 Khz to about 2 MHz to promote phonophoresis within tissue immediately adjacent the transducer;
control means for operating the transducer;
a power source operatively associated with the transducer for exciting the transducer to generate the ultrasound energy;
a reservoir disposed about and containing the transducer, the reservoir being adapted to contain a therapeutic substance in flowable form at body temperature, the reservoir being defined by an outer wall; and
at least one outlet formed through the outer wall to enable therapeutic substance to flow through the outlet into direct tissue contact

28. A device defined in claim 27 further comprising:
means communicating a source of pressure with the interior of the reservoir.

29. A device defined in claim 28 wherein the source of pressure is coupled with the reservoir through a lumen of an umbilical cord.

30. A device as defined in claim 3 further comprising:
means for replenishing the reservoir with a therapeutic substance from a location external of the body.

31. A device as defined in claim 30 further comprising a subcutaneous injection port and an umbilical cord extending from the injection port to the reservoir.

32. A device as defined in claim 3 further comprising means for attaching the device to soft tissue or bone.

33. A device as defined in claim 3 further comprising an acoustic matching layer disposed between the primary emission surface of the ultrasound transducer and the outermost surface of the device.

34. A device as defined in claim 33 wherein the outermost surface of the device comprises the outermost surface of the matching layer.

35. A device as defined in claim 3 wherein the ultrasound transducer is configured to emit ultrasound energy substantially omni-directionally.

36. A device as defined in claim 3 wherein the power source comprises an induction coil.

37. A device as defined in claim 3 wherein the source contains a therapeutic substance selected from the group consisting of chemotherapeutic compounds, genetic material, drugs, vitamins, amino acids, peptides, proteins, nucleic acids, DNA or RNA, anti-fungal agents, antibiotics, hormones, vitamins, anticoagulation agents, antivirals, anti-inflammatories, local anesthetics, radioactive agents, organic and inorganic compounds, contrast agents, therapeutic agents with short-life cycle, bubble nuclei, micro-spheres (substance encapsulated), combinations thereof and the like.

38. A device as defined in claim 5 wherein the source of therapeutic substance contains a carrier substance selected from the group consisting of saline, alcoholic saline, protein buffered saline, and hydrogel.

39. A method for locally delivering therapeutic substance to a patient comprising:
surgically implanting an ultrasound transducer in the immediate vicinity of the target tissue;
the transducer being configured to emit ultrasound energy at levels at which no substantial tissue necrosis occurs and in the frequency range of about 50 KHz to about 2 MHz to promote a phonophoretic effect oriented toward the target tissue;
providing therapeutic substance containing large or medium size molecules directly to the immediate vicinity of the target tissue;
activating the transducer to generate ultrasound to phonophoretically enhance the uptake by the target tissue of the therapeutic substance.

40. A method as defined in claim 39 wherein the molecules comprise genetic agents.

41. A method as defined in claim 39 wherein the substance comprises at least one of RNAi, sRNA and double strand RNA.

42. A method as defined in claim 39 wherein the step of providing the substance comprises:
surgically placing the substance together with the transducer immediately adjacent to tissue and juxtaposed with the substance located between the tissue and the ultrasound emission surface.

43. A method as defined in claim 42 further comprising containing the therapeutic substance in a reservoir and implanting the reservoir.

44. A method as defined in claim 43 wherein the transducer and reservoir are independent of each other and are implanted separately.

45. A method as defined in claim 43 wherein the transducer and the reservoir are disposed in the same unitary device.

46. A method as defined in claim 43 further comprising providing a transducer of small size and an outlet for therapeutic substance implantable together immediately adjacent target tissue; and
supplying power to the transducer from a remote source; and
supplying therapeutic substance to the outlet from a remote source.

47. A method as defined in claim 46 wherein at least one of the substance source and power source are implanted in a remote location within the patient and connected to the transducer and outlet by an umbilical cord.

48. A method as defined in claim 46 wherein at least one of the power source and source of therapeutic substance are disposed externally of the patient.

49. A method as defined in claim 43 wherein the reservoir comprises a pressure operated bladder.

50. A method as defined in claim 43 further comprising periodically replenishing the reservoir with therapeutic substance.
